(19) United States
(12) Patent Application Publication
(10) Pub. No.: US 2010/0288289 A1
(43) Pub. Date: Nov. 18, 2010

(54) AIRWAY DEVICE

(76) Inventor: Muhammed Aslam Nasir, Bedfordshire (GB)

Correspondence Address: HAYES SOLOWAY P.C., 3450 E. SUNRISE DRIVE, SUITE 140, TUCSON, AZ, 85718, US

(21) Appl. No.: 12/680,731

(22) PCT Filed: Sep. 29, 2008
(86) PCT No.: PCT/GB2008/050880

§ 371 Date: Jun. 23, 2010

(30) Foreign Application Priority Data
Sep. 29, 2007 (GB) 0719054.9
Mar. 28, 2008 (GB) 0805697.0

(51) Int. Cl. A61M 16/04; A61M 5/00; A61C 5/14

(52) U.S. Cl. 128/861; 128/207.14; 128/202.15

(57) ABSTRACT

An airway device for insertion into the trachea or bronchi of a human or animal, comprising an elongate flexible tube (10) having a distal end, a proximal end and a lumen therethrough, the device further comprising a cuff (11) located at or near the distal end of the flexible tube, wherein the cuff comprises an inner inflatable region (12) and an outer soft barrier region (13) adapted to prevent the walls of the airway coming into direct contact with the inner inflatable region when the cuff is in position and inflated.

### FIELD OF THE INVENTION

This invention relates to an airway device. It is particularly applicable, but in no way limited, to devices used in the administration of anaesthetic gases and oxygen to a patient breathing spontaneously, during intermittent positive pressure ventilation for a surgical procedure, during cardiopulmonary resuscitation or for ventilatory support in intensive care. The present invention also relates in particular to an endotracheal, endobronchial or a combined endotracheal/endobronchial airway device. More specifically, the present invention relates to an endotracheal airway device that is safer and easier to use than known endotracheal airway devices, that provides a greater degree of protection to the patient against aspiration, and to methods of fabricating such an airway device.

### BACKGROUND TO THE INVENTION

Endotracheal intubation is a common medical procedure and endotracheal tubes are used in many situations for ventilatory purposes. They are used in both human and animal surgery to provide passageways for anaesthetic gases into the lungs during routine or emergency surgery. They are also used as conduits for airway life support where patients have stopped breathing on their own (e.g., because of cardiac arrest, respiratory distress/arrest, seizures, and the like).

Many endotracheal tubes have an inflatable cuff system where the inflatable cuff creates a seal between the walls of the endotracheal tube and the inner surface of the tracheal wall, to prevent aspiration from the pharynx into the lungs and to allow spontaneous or positive pressure ventilation to be applied through the tube.

There are many types of endotracheal tubes currently used by medical practitioners but all of them carry significant disadvantages. There are two main problems that result from anaesthetising patients using one of the known endotracheal devices.

Firstly, under-inflation of the cuff will almost always encourage aspiration of mucus, blood stained mucus from the oral cavity or gastric regurgitate from the stomach, which is likely to accumulate over and above the cuff, encouraging a gravitational flow into the lungs which will cause infective or chemical aspiration pneumonitis. Secondly, over-inflation of the cuff will cause direct mucosal ischaemia leading to mucosal damage with long term consequences.

If aspiration is allowed into the lungs it can cause serious complications for a patient and is especially a problem for long-term intubation or on occasions when the patient has a full stomach. This is a major cause of post-operative problems in hospitals where the patients subsequently get lung infections after an operation.

In endotracheal tubes commercially available for use, when an inflatable cuff is inflated in situ, longitudinal streaks or folds in the surface of the cuff are formed. These channels allow any liquid which accumulates above the cuff and which usually comprises gastric juices, to flow past the cuff into the lungs. This can result in pulmonary oedema, fluid clogging in the respiratory tracts and subsequently prevent oxygen reaching the blood stream through the parenchyma of the patient's lungs, with fatal consequences. Even if this does not immediately lead to respiratory failure, the foreign fluid in the lungs can lead to chest infections or problems of oxygenation for the patient, requiring long term ventilatory support in intensive care along with all the other socio-economic consequences.

One way to reduce or prevent aspiration into the lungs is to inflate the cuff to such a pressure that these channels or streaks do not occur, or are at least reduced. In this way a better seal is formed between the cuff and the trachea and aspirate into the lungs is reduced. However, this leads to the second major problem associated with endotracheal tubes, because an increased cuff pressure puts a larger strain on the delicate tracheal tissue thus disrupting the blood supply and can damage it. During the process of intubation or post-intubation, the excessive trauma caused by the presently used endotracheal tubes can leave a raw traumatised surface on the pharynx, larynx and/or trachea leading to hospital acquired infections, pneumonia and other nosocomial infections.

Excessive pressure on the trachea can also result in erosion of the tube through the trachea into the oesophagus or into arteries or other vessels. This is usually fatal. The problem is exacerbated by the fact that anaesthetic gas (nitrous oxide) can diffuse into the cuff, expanding the air in the cuff during use, thus increasing the cuff's extra luminal pressure significantly, and as a result, put considerable pressure on the sensitive tissues of the trachea.

Therefore, on the one hand, in current designs there is a need for a cuff which forms a high pressure seal with the walls of the trachea to prevent aspirate entering the lungs and on the other hand the cuff needs to form a low pressure seal in order to prevent or at least reduce tracheal damage.

There are two main types of cuff design that try to balance these opposing requirements. The first is a high pressure, low volume cuff. These usually have a small profile and transmit high pressure onto the tracheal walls. They reduce the aspiration passing the cuff but can damage the trachea.

The second cuff is a low pressure, high-volume cuff. These usually have a larger profile. This spreads the cuff contact point over a larger area to minimise the pressure at any one point but they are more difficult to insert through the larynx and are less effective at preventing aspiration around the cuff. However they do reduce tracheal trauma. Because they have a higher compliance and are larger, it is much easier and more likely for channels to form in the cuff once inflated and therefore aspirate can easily pass around them.

Accordingly, it is an object of the present invention to overcome or mitigate some or all of these problems.

### SUMMARY OF THE INVENTION

Accordingly, according to a first embodiment, there is provided an airway device for insertion into the trachea or bronchi of a human or animal comprising an elongate flexible tube having a distal end, a proximal end and a lumen therethrough, the device further comprising a cuff located at or near the distal end of the flexible tube, wherein the cuff comprises an inner inflatable region and an outer soft barrier region adapted to prevent the walls of the airway coming into direct contact with the inner inflatable region when the cuff is in position and inflated.

The airway device according to the present invention provides major advantages over prior designs. The design is suitable for, among other things, use as or along with an endotracheal or an endobronchial tube. The outer soft barrier region can serve a dual purpose. Because of its softness, it does not damage the tissues surrounding the cuff when it is inflated. Therefore, the cuff provides a sufficient seal without damaging the soft delicate tissue around the device.

Furthermore, the outer barrier layer reduces the chance of leakage of fluid around the cuff because the barrier layer does not form the longitudinal folds that allow aspiration around the cuff.

Both of these advantages individually provide a significant advance over known devices and in combination provide an endotracheal device that can reduce both tracheal trauma and post-operative complications.

The cuff is located towards the distal end of the device. It is generally situated above the distal end but could be at the distal end itself. In this latter case the tube would not protrude substantially beyond the end of the cuff.

The cuff comprises an inner inflatable region and an outer soft barrier region. The barrier region is effectively a protective layer to protect the patient and to improve the seal formed between the device and the airway. The soft barrier region can either be formed by a separate outer barrier layer over the inner inflatable region or alternatively, the inner barrier could comprise an outer soft barrier region as part of its outer shell. The soft barrier region is formed such that it cushions and protects the walls of the airway from the inflatable region.

The outer barrier region can be thought of as a layer that sits over the inner inflatable region and protects the patient from the damaging effects of the inflatable region of the cuff, when the cuff is inflated to a degree sufficient to reduce or prevent aspirate entering the lungs past the device's cuff.

Preferably the outer barrier region surrounds substantially the entire external surface of the inner inflatable region. Thus the entire inflatable region can be protected by the barrier or protective layer.

Alternatively, the outer barrier region may only surround the area or areas of the inner inflatable region that come into contact with the trachea/bronchus when the cuff is in position and inflated.

Thus the outer barrier can completely surround the entire inflatable region or alternatively can only surround the regions that come into contact with the trachea, for example the edges of the cuff. This reduces the need to form the barrier layer over the entire cuff.

A major advantage of the soft outer barrier region is that its properties, e.g. its hardness or its propensity to channel formation, do not change as the cuff is inflated. This is in marked contrast to the prior art inflatable cuffs as the outside surface of the cuff is entirely dependant on the level it is inflated by. With a minimal inflation the surface forms channels and with a higher inflation level, the surface is hard and can damage the airway tissue. The outer surface of the soft barrier layer of the present invention is not affected by the level of inflation of the device and therefore always forms an effective seal without channels and does not damage the tissue of the patient.

It is possible to make the cuff entirely out of the material for the outer soft barrier region. In this instance the inner surface of the soft barrier layer effectively defines the inner inflatable region and the outer surface of the soft barrier layer forms the effective seal and prevents tissue damage. It is necessary to select a barrier material that is substantially air tight to allow the cuff to be inflated.

Preferably the outer barrier region is formed from a material with a Shore hardness on the A scale of 40 or less and more preferably between 0 and 30, and most preferably between 000 and 12. The shore hardness of the outer barrier region/layer is an important feature of the invention. Where feather flanges are present these are typical of shore hardness 000 to 10.

Preferably the outer barrier region is overmoulded over the inflatable region. This allows the inflatable region of the cuff to be formed first from a harder, less compliant material and then the softer, more compliant barrier region can be formed on top of the inflatable region.

In a particularly preferred arrangement, the outer barrier region further incorporates one or more feather like flange(s) having a base attached to the outer barrier region, and a tip pointing caudally.

The feather like flanges help to improve the seal between the cuff of the airway device and the trachea. Preferably the feather like flanges extend around substantially the entire circumference of the cuff, thus helping to further seal the entire airway.

In one embodiment, the feather like flange(s) are or cilia like projections that extend from the outer barrier region in a direction normal to the longitudinal axis of the airway device. Thus, the flanges extend out at substantially 45 degrees from the body of the outer barrier region.

Alternatively, the tip of the feather like flange(s) may be nearer to the proximal end of the tube than the base of the feather like flange(s). In this instance, the flanges point or extend away from the body of the barrier region towards the proximal end of the device, preferably at an angle of about 15-75 degrees and most preferably 45 degrees. This arrangement aids insertion of the device and also help to collect the aspirate by encouraging it to pool above the cuff rather than sitting at the edges of the cuff and working its way past over time.

When children need to be intubated, it is not advisable to use an inflatable cuff and presently, anaesthetists use a standard airway tube without a cuff. This obviously does not provide a good seal with the airway of the child but because their airways are softer, smaller, under-developed and more easily damaged, it is more preferable than the inevitable tissue damage that results from using an inflatable cuff.

The present invention provides for the first time a way of both increasing the seal formed with, and reducing the damage from, an airway device. By using an inflatable region with a small profile with an outer barrier layer the airway device will not damage the soft tissue of the child. Alternatively, the device could be provided without an inner inflatable region and have and airway tube with a soft barrier layer. Thus, the device is safer to use as it has a soft outer region and will also provide a greater seal because it can come into a stronger contact with the walls of the child's airway, thus forming a better seal.

Therefore, for the first time, there is provided an airway device that is non-cuffed, for a child that both reduce the trauma to the child's airway whilst increasing the seal formed. It may of course be desirable to construct a device that only addresses one of the above issues. This is likely to be the case when damage could be catastrophic, or alternatively if a seal is absolutely necessary. This is achieved by coating all or at least a part of the airway tube with an outer barrier layer, with or without feather-like flanges, to prevent the tube damaging the soft tissue of the airway and to provide a greater seal. There could also be provided a small inner inflatable region.

According to a second aspect of the present invention there is provided an airway device for insertion into the trachea or bronchi of a human or animal, comprising an elongate flexible tube having a distal end, a proximal end and a lumen therethrough, the device further comprising a cuff located at or near the distal end of the flexible tube, wherein, in use, the cuff is so sized, shaped and positioned in relation to the tube such that it engages the walls of the airway in a plane non-perpendicular to the longitudinal axis of the airway at the cuff.

In addition to the physical trauma of the cuff against the trachea, tracheal wall pressure from the cuff can impede blood flow through the trachea and into the tracheal mucosa. By ensuring that the cuff engages the walls of the trachea in a plane non-perpendicular to the axis of the trachea at the cuff, the blood can always flow through the tissue of the trachea around the cuff because there is always a path through the tissue that is not in contact with the cuff. This angled or offset cuff is in contrast to a cuff that substantially extends in a plane substantially perpendicular or normal to the trachea. In the latter example, the cuff reduces or cuts off entirely (in the case where the cuff pressure is too high) the blood flow from the region below the cuff to the region above the cuff and vice versa. In the cuff of the present invention, there is always a path for the blood to flow because in a given plane, there is always a portion of the trachea that is not being pressed or contacted by the cuff.

Preferably the cuff extends from the tube in a plane non-perpendicular to the longitudinal axis of the tube at the cuff. The cuff can be thought of as an offset disk, sausage, donut or torus.

It is also possible to include two offset cuffs arranged in an x-shaped or overlapping configuration. This effectively provides a double barrier to prevent aspirate but does not completely surround a cartilage ring causing necrosis.

According to a third aspect of the present invention there is provided an airway device for insertion into the trachea or bronchi of a human or animal, comprising an elongate flexible tube having a distal end, a proximal end and a lumen therethrough, wherein the tube comprises a first substantially straight tubular region at the proximal end of the device and a second tubular region at the distal end of the device, the second tubular region comprising a cuff and projecting at an angle of 15-70 degrees from the longitudinal axis of the first tubular region.

Up to now, endotracheal tubes have been generally curved, straight or preformed. The curve has been employed to allow the device to conform to the orientation of the larynx in relation to the mouth of the patient.

However, this causes problems when inserting the device through the patient's mouth because during laryngoscopy, the curve of the tube substantially occludes and obstructs the view of the back of the mouth from the sight of the anaesthetist thereby making it difficult to align and direct the device into the trachea.

A laryngoscope, which is a device used to directly visualize the larynx to aid in the insertion of an endotracheal tube through the vocal cords is often used to aid insertion into the larynx but its use is severely hampered by the curved nature of the tube because of this occlusion and in practice intubating a patient is often a difficult procedure. The advantage of the first substantially straight region together with the second tubular region extending at an angle from the longitudinal axis of the first tubular region is that there is no curve to prevent the laryngoscope from viewing the entire mouth and larynx. Therefore once the second tubular region has entered the pharynx or larynx, the straight first tubular region does not affect the use of a laryngoscope and allows the anaesthetist a clear view to aid insertion of the device. This minimises movement of the device in the trachea and therefore reduces trauma and damage to the soft tissue.

This offset tube region can be considered an L-shaped or golf stick cuff, although the specific angle will not necessarily be 90 degrees.

According to a fourth embodiment of the present invention there is provided an airway device for insertion into the trachea or bronchi of a human or animal, comprising an elongate flexible tube having a distal end, a proximal end and a lumen therethrough, the device further comprising a cuff located at or near the distal end of the flexible tube, wherein the cuff comprises a mechanical extending means operable to move the cuff from a non-extended position wherein the cuff does not engage the walls of the airway and an extended position wherein the cuff engages the walls of the airway.

For the first time there is provided an airway device that uses a mechanical extending means to ensure that a fluid tight seal is formed between the cuff and the airway. This has a number of advantages over the inflatable cuffs in the prior art. Inflatable cuffs are subject to over inflation due to an influx of gas diffusing into the inflatable region. Furthermore, there is a need for additional equipment to maintain the cuff at the correct pressure. Also, cuffs of this design are expensive to manufacture and require complicated tooling and components. For the first time, there is provided a cheap, easy way to move the cuff into an engaging position.

The mechanical extending means is preferably enclosed within the cuff. Therefore the potentially sharp or hard edges of the mechanical extending means cannot come into contact with the airway and damage the tissue. The degree of enclosure will depend on what is necessary to both form a good seal and protect the patient.

Preferably the mechanical extending means comprises an extending portion and means to move the extending portion between a non-extended and an extended position.

Thus, when the cuff is being inserted, the extending portion is flush with the tube and the device can easily be inserted into position. Once it is in position, the extending portion is moved into an extending position by the extending means.

Preferably the extending portion is pivotally mounted with respect to the tube. One way of achieving the mechanical extending means is to have the extending portion pivotally mounted with respect to the tube. Therefore when the portion is extended, it will pivot out from the tube and will therefore give the cuff a wider profile that will engage with the walls of the airway. The mechanical extending means sits within the cuff and pushes it outwards as it pivots away from the tube.

In a particularly preferred embodiment the extending portion is pivotally mounted at or near the distal end of the cuff such that the proximal end of the cuff extends further from the tube than the distal end of the cuff. This allows the proximal end of the cuff to be open to allow aspirate to collect within the cuff. Thus, once the cuff is in an extended configuration, it is shaped like an inverted umbrella, allowing the aspirate to collect in the cuff. The aspirate is then not pooling around the edges of the cuff and can be collected by a suction tube.

Preferably the means to move the extending portion moves longitudinally between the tube and the extending portion, forcing the extending portion to extend from the tube. If the extending portion is pivotally attached at the distal end of the tube and the means to move the extending portion is between the extending portion and the tube then downward movement forces a wedge between the tube and the extending portion, forcing them apart. This provides an easy way of extending the cuff and does not require complicated mechanics.

Preferably the means to move the extending portion comprises a sleeve slideably mounted with respect to the outer surface of the tube. Alternatively, any device that can extend between the tube and the extending portion could control it. In addition, hydraulics could be employed to extend the extending portion.

Advantageously the sleeve comprises ridges to push the extending portion from the tube. This ensures that downward motion extends the cuff to a sufficient degree.

In a particularly preferred embodiment the extending portion comprises a plurality of fingers. Therefore as the fingers extend outwards they push the cuff out to engage with the airway wall. Again, the analogy with an upside-down umbrella is a good one, equating the fingers with the mechanical arms of the umbrella.

Optionally the extending portion is hinged.

Preferably the extending portion is pivotally mounted both with respect to the tube and the means to move the extending portion. Therefore as the cuff extends, the fingers can be directly attached to both the tube at the distal end and the means to extend the extending portion at the other end. Hinges in the fingers allow them to extend outwards.

Obviously a single extending portion encircling the tube could also be employed.

Particularly preferably, the cuff further comprises an outer soft barrier region. Therefore, the soft cuff as explained above can be usefully employed with this embodiment. The extending portion must be covered with some form of barrier to enable it to form a seal and not damage the airway but a device incorporating an outer soft barrier as outlined above will provide the best seal and the safest use.

Preferably the outer soft barrier region is not attached to the airway device at its proximal end. This allows the aspirate to collect within the cuff. Particularly preferably the outer soft barrier region is not attached to the mechanical extending means at or near its proximal end.

The above embodiments are suitable for an endotracheal tube, an endobronchial tube or a combined endotracheal and endobronchial tube.

According to a fifth aspect of the present invention there is provided a device for insertion into a lumen of a human or animal, comprising an elongate flexible tube having a distal end, a proximal end and a lumen therethrough, the device further comprising a cuff located at or near the distal end of the flexible tube, wherein the cuff comprises an inner inflatable region and an outer soft barrier region adapted to prevent the walls of the lumen coming into direct contact with the inner inflatable region when the cuff is in position and inflated.

According to a sixth aspect of the present invention there is provided a device for insertion into a lumen of a human or animal, comprising an elongate flexible tube having a distal end, a proximal end and a lumen therethrough, the device further comprising a cuff located at or near the distal end of the flexible tube, wherein, in use, the cuff is so sized, shaped and positioned in relation to the tube such that it engages the walls of the lumen in a plane non-perpendicular to the longitudinal axis of the lumen, at the cuff.

According to a seventh aspect of the present invention there is provided a device for insertion into a lumen of a human or animal, comprising an elongate flexible tube having a distal end, a proximal end and a lumen therethrough, the device further comprising a cuff located at or near the distal end of the flexible tube, wherein the cuff comprises a mechanical extending means operable to move the cuff from a non-extended position wherein the cuff does not engage the walls of the lumen and an extended position wherein the cuff engages the walls of the lumen, the cuff further comprising an outer soft barrier region.

The advantages described above can either be used individually or in combination with each other. For example, the barrier region can be applied to existing inflatable cuff designs or alternatively used in combination with the offset, angled cuff or mechanical extension means. Furthermore, the offset tube region can be used with existing cuff designs or with the new designs outlined in the present invention.

An integral, unitary or parallel suction channel is preferably provided above the cuff region to aspirate the secretions or any regurgitate.

Pressure sensors are preferably incorporated in the soft barrier layer in order to measure and monitor the seal pressure in between the cuff and the tracheal wall.

Another arrangement of the cuff is described herewith that the cuff is an umbrella shaped but non-inflatable, where the conical part of that umbrella shape faces distally and the widest part proximally where the outer surface of the cuff of this arrangement fully or partly covered with the soft barrier layer.

It will be understood that the present invention extends to cover methods of manufacturing the airway devices described herein, to methods of anaesthetising and methods of aspirating a human or animal subject, and to methods of treatment and diagnosis resulting from the use of such devices.

### BRIEF DESCRIPTION OF THE DRAWINGS

The invention will now be described, by way of example only, with reference to the accompanying drawings in which:—

FIG. 1 illustrates in plan view of a first embodiment of the present invention showing an inflatable cuff in an un-inflated configuration;

FIG. 2 shows a plan view of the device in an inflated configuration;

FIG. 3 shows a plan view of the device with a barrier region only over the areas that come into contact with the tracheal wall;

FIG. 4a shows a plan view of a second embodiment of the present invention showing an offset cuff in an inflated configuration, in the trachea;

FIG. 4b shows a plan view of a second embodiment of the present invention showing an offset cuff in an un-inflated configuration;

FIGS. 5a and 5b show plan views of endotracheal tubes having two offset cuffs;

FIG. 6a shows a plan view of a third embodiment of the present invention showing an airway device with an offset tube;

FIGS. 6b and 6c show the embodiment of FIG. 6a including a gastric tube channel and both a gastric tube channel and a suction tube respectively;

FIG. 7 shows another plan view of a pediatric airway device;

FIGS. 8a and 8b show a plan view of a fourth embodiment of the present invention showing a mechanically extendable cuff in an un-extended and extended configuration;

FIGS. 8c and 8d shown an embodiment similar to that in FIGS. 8a and 8b in which the open face of the cuff is directed downwards into the trachea;

FIGS. 9a and 9b show a plan view of an alternative fourth embodiment of the present invention showing a mechanically extendable cuff in an un-extended and extended configuration;

FIGS. 9c and 9d show an embodiment similar to that of FIGS. 9a and 9b in which the open face of the cuff is directed downwards into the trachea;

FIGS. 10a and 10b illustrate an embodiment in which the cuff is extended by way of a small inflatable collar;

FIGS. 11 to 13 inclusive shown embodiments in which the cuff is inflatable but where there is an air gap between the airway tube and the cuff where it contacts the trachea;

FIGS. 14a and 14b illustrate an embodiment wherein the cuff is expanded by means of a mechanical extension means;

FIGS. 15a and 15b illustrate a cuffed endotracheal tube with a butterfly-shaped cuff;

FIGS. 15c and 15d show a version of a butterfly-cuffed tube incorporating a gastric tube passageway and a drainage tube;

FIGS. 16a and 16b illustrate a cuffed endotracheal tube in which the airway tube has a reduced diameter in the region of the cuff such that, in its non-extended state, the cuff is contained within the general outer profile of the airway tube;

FIGS. 17a and 17b illustrate an alternative embodiment in which the cuff is extended by way of a small inflatable collar and illustrates an alternative distal tip;

FIGS. 18a, 18b and 18c illustrate alternative distal tips which can be employed with any of these embodiments.

### DESCRIPTION OF THE PREFERRED EMBODIMENTS

Embodiments of the present invention are described below by way of example only. These examples represent the best ways of putting the invention into practice that are currently known to the applicant although they are not the only ways in which this could be achieved.

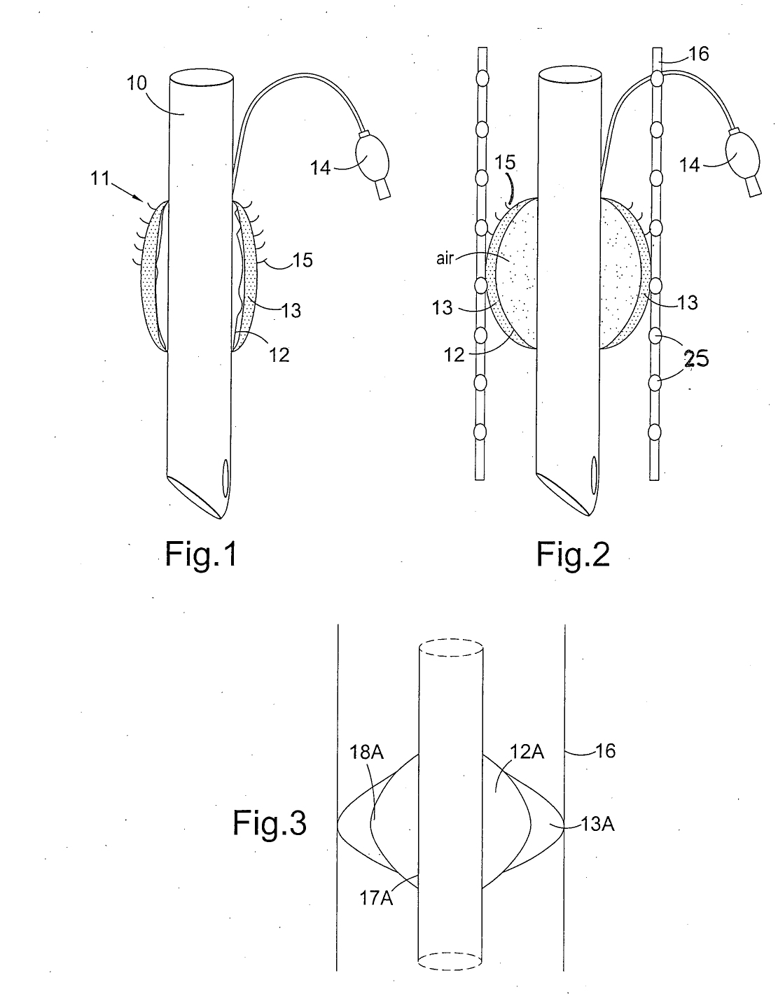

FIG. 1 shows an endotracheal device according to a first embodiment of the present invention. The device is shown in an un-inflated or deflated position. The device comprises an airway tube 10 and a cuff 11. The cuff, or in this case cuff assembly, is made up of an inner inflatable region 12 and a soft outer barrier region 13 together with means to inflate the inner inflatable region 14. The inner region 12 is similar to the inflatable cuffs known in the prior art although it has a smaller profile to accommodate the presence of the outer barrier region. Typically, the inner barrier region will expand enough to allow a good seal to form between the outer barrier region and the walls of the trachea. The inner inflatable region can be of a lower compliance than would otherwise be desirable as it does not come into contact with the trachea, in use, and therefore it will not damage the soft tissue. In addition, a higher cuff pressure can be applied because, again, it does not come into contact with the walls of the trachea.

It is also possible to use a low volume/low pressure cuff, this is in contrast with prior art cuffs which require a low volume/high pressure or alternatively a high volume/low pressure. In the present invention, a sufficient enough volume of air is inflated into the cuff to stop any gaseous leak between the outer barrier region and the tracheal wall or, if cuff pressure sensors or cuff pressure monitors are used, then a pressure of not more than 25 mmHg.

Because of the outer barrier region, it is possible to use a smaller tube size for a given patient. For example, an adult male will typically use a size 9 tube (9 mm internal tube diameter). With an airway device according to the present invention it is possible to reduce the size of the tube (e.g. to a size 8 tube), which would allow the barrier layer to be half a millimetre thicker on each side of the cuff. This makes the device more compliant and will reduce the trauma to the patient. The slight reduction in the tube size will not significantly affect the flow characteristics of the gases passing through the device.

The outer soft barrier layer can be formed from any suitable soft plastics material. By way of a preferred softness (hardness) range, on the Shore A scale of Hardness, a hardness of less than 20 for the barrier layer is optimum, with a preferred range of 0 to 12 and a particularly preferred range of 000 to 10. With regards to the body of the endotracheal tube, by way of a preferred range, a value on the same scale of between 20 to 80 is preferred, with a particularly preferred range of 40 to 60. The softness of the cuff can be further adapted by foaming the barrier layer material or by forming cavities or channels in the outer barrier layer although care needs to be taken to ensure that these channels do not allow aspiration to flow past the device. If the channels are formed in a lateral direction, that is in a direction substantially normal or perpendicular to the longitudinal axis of the airway tube, as opposed to a longitudinal (top to bottom) direction, then this problem is avoided.

It is now possible to produce plastics materials with a Shore hardness of 00 or 000. It is intended that these very soft materials are included in the definition of Shore hardness 0 on the A scale. In fact, materials having a shore hardness of 00 or 000 (‘triple zero’) are particularly effective as a barrier layer for covering the cuff structure where it is designed or intended to contact the trachea of the patient. It is also possible that the main structure of the cuff can be formed from such a material. It will be understood that, if this is the case, some form of cuff supporting structure will be required. Such a structure could be formed by, for example, a plurality of struts, arms, fonds or other braces which, singly or in combination, form the necessary curved or hemi-elliptical shape, umbrella-shaped or substantially frustoconical shaped assembly (see below).

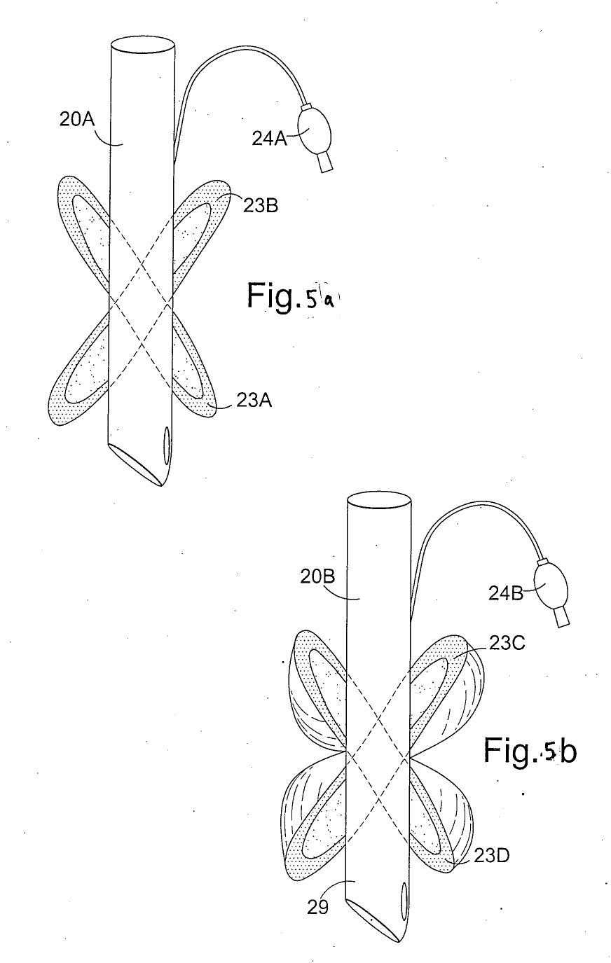

This very soft material may also be used to coat or form the distal tip of the endotracheal tube. This is shown as region 29 in FIG. 5b. The region of soft material in this example extends beyond the Murphy Eye, but this is not essential. Methods for overcoating one plastic with another are well known, as are methods of moulding a softer plastic onto a firmer plastic as part of an otherwise continuous structure. If the soft tip is overmoulded then a very soft plastics material, such as one with a Shore hardness of 000 may be used. If the tip itself is formed of a soft material then Shore hardness may be greater than 000, and towards the upper end of the range quoted above.

The concept of using a soft tip region, whilst not shown in every illustration, is applicable to all aspects and embodiments of the present invention.

In fact, it should be understood that the various embodiments of the invention described herein can be applied in any suitable combination, and are specifically not limited to the context of the example in which they are described.

The device itself may be constructed from any suitable plastics material as selected by the materials specialist. Latex-free medical grade silicone rubber is one preferred material. The cuff should be soft in texture to avoid undue damage to the surrounding tissue. Other suitable materials for construction of this type of device include, but are not limited to, Poly Vinyl Chloride (PVC), Thermoplastic Elastomers such as the styrenic block copolymers (e.g. Styrene Butadiene Styrene (SBS), Styrene Ethylene Butylene Styrene (SEBS)), and Thermoplastic Olefin Blends (TPO), Thermoplastic PolyUrethanes (TPU), Copolyester (COPE), Polyether Block Amides (PEBAX) and foamed versions thereof, where appropriate. This listing is provided by way of example only, to illustrate the wide range of polymers which may be used.

A further important factor involved in the choice of a suitable material is transparency. Ideally the material or materials of construction should be substantially clear or transparent. This enables the anaesthetist or operator to see the inner lumen of the airway to check for blockages or other problems. Such transparent materials are known to the materials specialist.

Where the cuff is inflatable, or contains an inflatable component, the cuff will preferably be constructed of such a material that will not allow nitrous oxide (anaesthetic gas) to diffuse through the material to any significant amount so that the extra luminal pressure is kept constant. It follows therefore that the cuff should be substantially impermeable to the fluid with which it is filled and to anaesthetic gases. In this embodiment, this can either be the material for the inner inflatable region, or it can be the material forming the outer soft barrier region, or both.

The outer barrier layer can also incorporate flanges, or feather flanges 15. These help to improve the seal between the device and the walls of the trachea or bronchi. In the embodiment shown in FIG. 1, substantially annular, thin, flexible featherlike flanges 15 have been introduced onto the outer surface of the outer barrier region. They are preferably formed as an integral part of the moulding and, because of the very soft nature of the material used to form the outer region of the cuff, these flanges are particularly soft and pliable. Their purpose is to make allowance for any individual patient variation in the airway and to contribute to forming an efficient and effective seal between the cuff and the tracheal wall.

The feather-like flanges can either be discontinuous structures or can extend radially around substantially the entire outer perimeter of the cuff, thus forming a seal with the walls of the airway. This has the added advantage that it helps to prevent aspirate from draining past the cuff because it provides an effective barrier to any liquids. In the example shown the flanges extend from the outer barrier layer and are pointed towards the proximal end of the device. This allows for ease of insertion because the flanges will be pushed flat against the outer barrier layer as it is moved into position and also helps prevent aspirate from moving past the cuff. Because of the soft, compliant nature of the flanges, they do not catch on the patient's soft tissue when it is removed from the airway with the cuff fully deflated. Whilst not always shown, these feather like flanges can be applied to all the embodiments and aspects described herein.

FIG. 2 shows the cuff of FIG. 1, in an inflated position. It shows the outer barrier region in direct contact with the walls of the airway 16. The inner barrier layer does not contact the walls of the airway as it is protected by the outer barrier layer. Thus, the delicate tissue is not damaged, or at least the damage is reduced, because the soft barrier layer is made from a very pliable material that will not cause irritation, excessive compression trauma or damage.

The outer barrier layer is not inflated or deflated and remains a substantially constant shape. It is believed that in some instances with prior art devices, the inflatable cuff is not fully inflated by the operator in order to minimise damage. This means that the outer surface of the cuff is not fully taut and therefore channels will inevitably form thereon. For example, if a beach ball is only partially inflated, there are folds and channels on the outer surface but when it is fully inflated, the outer surface is free from wrinkles. It is necessary to have an outer surface area of the cuff to exceed the area it is to fill, in order to get a compliant cuff and to account for variations in a patient's airway thickness. This excess material also contributes to the longitudinal folds and channel formation.

The airway device of the present invention avoids this because the outer surface is already formed in its substantially final configuration, free from wrinkles or channels. Therefore, once it is engaged against the walls of the trachea due to inflation of the inner inflatable region, the outer surface will be free from wrinkles or longitudinal channels (as would be found in a very high pressure cuff), but with a high compliance surface that minimises or avoids altogether, damage to the walls of the airway.

FIG. 3 shows an airway device wherein the outer barrier region 13A only extends over the part of the inner inflatable region 12A that would otherwise come into contact with the walls of the airway 16A. This means that the outer barrier region does not need to extend over the entire outer surface of the inflatable region as some parts of the inner inflatable region 17A, will never come into contact with the walls of the area whereas the edge areas of the inner inflatable region 18A will need to be protected.

Therefore, in order to minimise manufacture costs, it is possible to only overmould the outer barrier region over the central area of the inner inflatable region. Generally, it is envisaged that any area that might come into contact with the walls of the airway will be coated with an outer barrier layer.

It will be appreciated that this invention is applicable to endotracheal devices, endobronchial devices or any device that is required to be inserted into a lumen in a patient, where a seal is required but damage to the soft tissue is to be prevented.

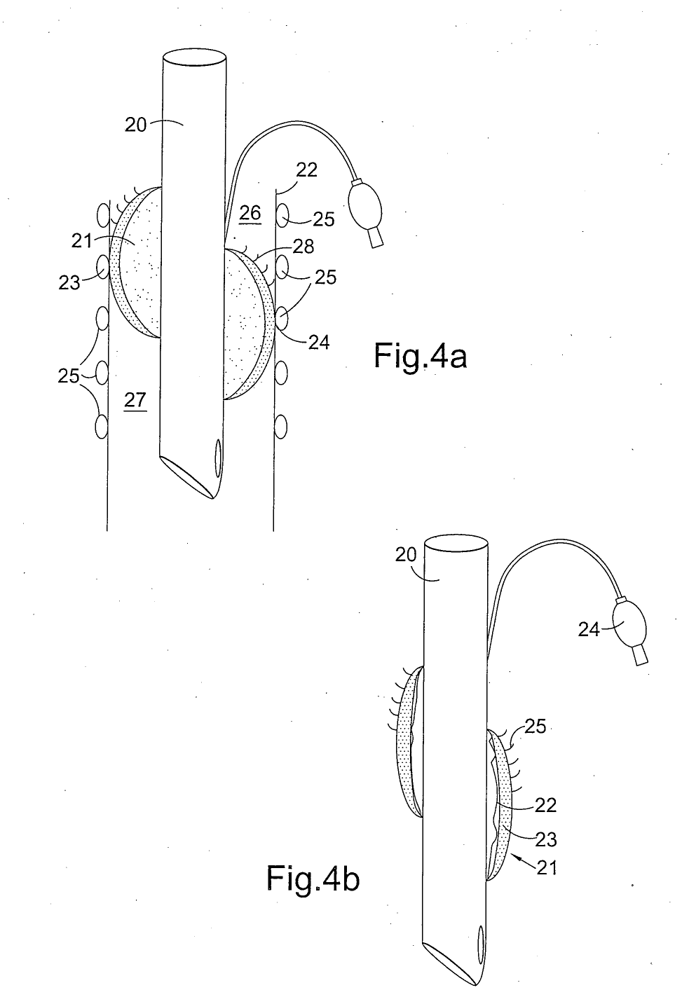

FIGS. 4a and 4b shows a second aspect of the present invention. FIG. 4a shows an airway tube 20 with an offset or angled cuff 21 in an inflated configuration, that is to say offset or angled with respect to the longitudinal axis of the airway tube. It can be seen that the cuff assembly 21 is arranged with respect to the walls of the airway 22 in a plane non-perpendicular to the longitudinal axis of the trachea at the cuff. Therefore, in this cross-section, the cuff engages the wall of the airway at region 23 and at region 24. It should be appreciated that the airway is substantially cylindrical and the cuff engages the entire inner circumference of the airway. However, instead of engaging the walls of the airway in a plane perpendicular to the longitudinal axis of the trachea at the cuff, the engagement is offset. This means that no part of the airway engages the cuff around a particular entire inner circumference of the airway while the cuff is in situ.

The cartilages 25 of the trachea vary from sixteen to twenty in number: each forms an imperfect C-shaped ring, which occupies the anterior two-thirds or so of the circumference of the trachea, being deficient behind, where the tube of the trachea is completed by fibrous tissue and unstriped muscular fibres. The cartilages are located horizontally one above each other, separated by narrow intervals. They measure about 4 mm. in depth and 1 mm. in thickness. Their outer surfaces are flattened in a vertical direction, but the internal surfaces are convex, the cartilages being thicker in the middle than at the margins.

The cartilages are enclosed in an elastic fibrous membrane, which consists of two layers; one, the thicker layer, passing over the outer surface of the ring, the other over the inner surface: at the upper and lower margins of the cartilages the two layers blend together to form a single membrane, which connects the rings one with another. They are thus invested by the membrane. In the space behind, between the ends of the rings, the membrane forms a single layer.

In addition to the fibrous membrane, there is also a mucous membrane, which is continuous above with that of the larynx, and below with that of the bronchi. It consists of areolar and lymphoid tissue, and presents a well-marked basement membrane, supporting a stratified epithelium, the surface layer of which is columnar and ciliated, while the deeper layers are composed of oval or rounded cells. Beneath the basement membrane there is a distinct layer of longitudinal elastic fibres with a small amount of intervening areolar tissue. The submucous layer is composed of a loose meshwork of connective tissue, containing large blood vessels, nerves, and mucous glands; the ducts of the latter pierce the overlying layers and open on the surface.

The offset cuff of this embodiment ensures that no cartilage ring has the cuff pressed against its entire inner circumference when the cuff is inflated. When a cuff is inflated and presses against the soft membranes and tissue on the outside of the cartilage, it restricts blood flow into the compressed area. This eventually causes necrosis of the tissue, which is starved of blood and therefore oxygen. By having an offset cuff, there is always an area on each cartilage ring that is not compressed, which allows blood to flow through this area and helps to provide blood to the compressed areas.

Thus, the offset cuff prevents severe tissue damage or necrosis caused by the compression of the entire membrane surrounding the inner surface of the cartilage by ensuring that a portion of each cartilage is not compressed.

The offset cuff also reduces the expansion force acting on a given area of the airway by spreading out this force over a greater area. When the cuff engages the walls of the airway in a normal plane to the airway, there is a lateral force acting in all outward directions on a specific portion of the airway. This stretches the muscle and causes damage to the expanded area of the airway. In comparison, the offset cuff ensures that a force acts in one direction on one area of the airway 23 and in the opposed direction on another, different, area of the airway 24.

The offset cuff also allows for the gastric juice or aspirate to pool and collect in one region 28, which has a number of advantages. Firstly, if a suction tube is used to collect aspirate whilst the device is in position, it can be positioned into region 28 and will therefore collect the entire aspirate, as it will naturally pool around the suction tube. Secondly, the cuff can be designed to collect the aspirate, for example in a recess or depression. This means that less aspirate will be able to pass the cuff into the lungs of the patient as more of it will be contained in a safe region, being the depression, and will not be working its way past the cuff.

The offset cuff can incorporate any or all of the features relating to the outer barrier layer, including the feather flanges.

A variety of shapes can be used to achieve the offset nature of the cuff. For example, the cuff can be shaped like a donut, angled from a plane normal of the longitudinal axis of the tube. Alternatively, the cuff could extend from the tube in a plane substantially normal to the longitudinal axis of the tube but be arranged such that the regions of the cuff that contact the walls of the airway are offset so that they do not completely surround a cartilage ring or rings.

FIG. 4b shows a deflated version of the offset cuff.

FIGS. 5a and 5b show alternative embodiments of the offset cuff principle, where there are two donut shaped cuffs 23A, 23B, 23C, 23D in a crossed arrangement. This arrangement increases the barrier area of the cuff assembly in contact with the trachea to reduce aspirate passing the cuff, as there is effectively two layers of barrier. Care needs to be taken to ensure that the arrangement does not completely surround a cartilage ring or rings. The size of each cuff, and in particular the size of the area on the perimeter of the cuff that contacts the trachea, may be determined by the materials expert. An embodiment with a larger contact area is shown in FIG. 5b. That is to say, the inflatable lobes of the cuff are larger than those shown in FIG. 5a. the airway tube is shown as 29.

The angle the two cuffs intersect each other does not have to be 90 degrees.

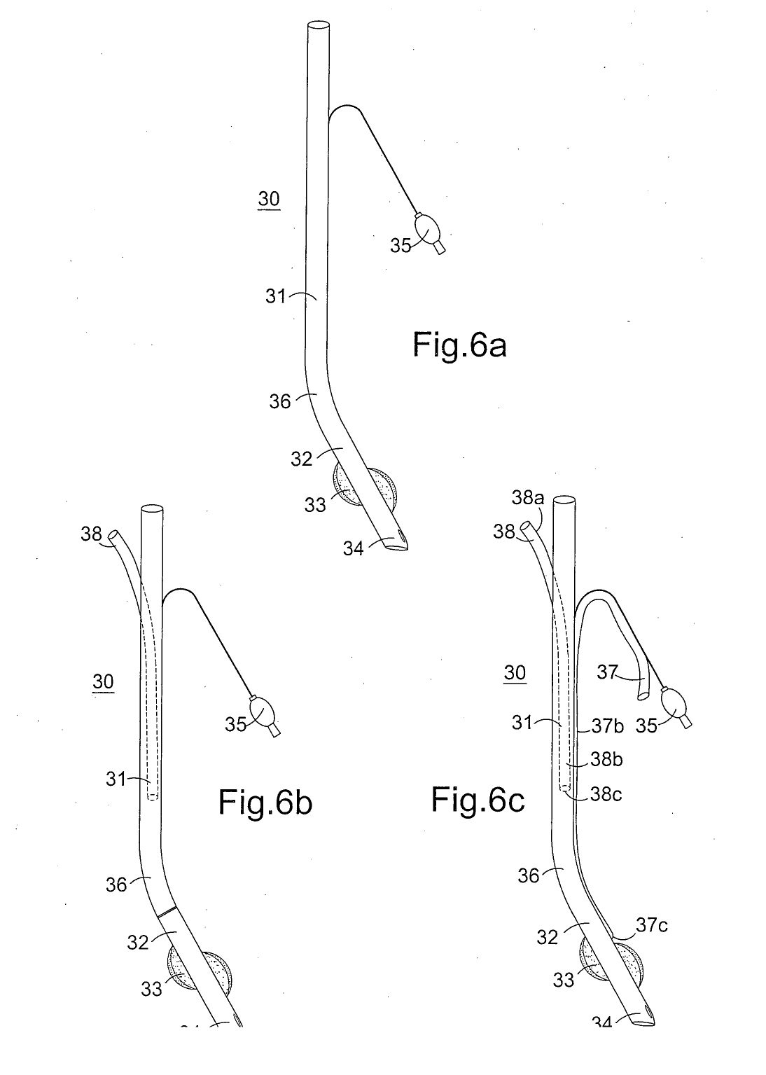

FIG. 6 shows a third embodiment of the present invention. It shows an airway tube 30 which has a first substantially straight tubular region 31 and a second tubular region 32 that extends at an angle from the longitudinal axis of the first tubular region, the second tubular region further comprising a cuff assembly 33 and incorporating the distal end of the tube 34. An inflation means 35 for the cuff assembly is also shown. An angled airway tube can be incorporated into any of the embodiments described herein.

The second tubular region 32 can be separated from the first tubular region 31 by a section 36 that enables the offset angle of region 32 to be adjusted. This can take the form of a concertina arrangement or a material that can be adjusted into a particular conformation and remain in that conformation during the process of intubation. As soon as the intubation is accomplished the lower tubular part adjusts its shape to the longitudinal axis of the trachea. This arrangement not only helps improve the laryngoscopy view but also help ease of intubation thus avoiding the trauma of laryngoscopy and intubation.

The offset tube can be used in isolation or in conjunction with any or all of the concepts outlined above relating to the outer barrier region or the offset cuff, or any of the concepts described below. As can be seen from FIG. 6, the cuff used in this example is offset.

FIGS. 6b and 6c show additional, optional features that can be incorporated into all aspects and embodiments of the present invention. A gastric tube passageway 38 is provided which extends from the proximal end of the device 38a to a point 38b partway along the airway tube and towards, but short of, the distal tip of the airway tube. Whilst the proximal end of this gastric tube passageway is external to the airway tube, it is preferred that the length of this passageway which is continuous with the airway tube is internal to the airway tube. However, it is equally possible that the passageway may run external to the airway tube.

The distal exit of the passageway 38c exits through the wall of the airway tube and external to the airway tube at a point that a gastric tube would naturally be directed towards the oesophagus.

A suction tube 37 is also or alternatively provided. This suction tube 37 extends from the proximal end of the device to a point 37C immediately above the cuff 33. Both ends of this tube are open and the proximal end is adapted to be connected to a syringe or other suction device to remove any fluid that collects above the cuff. In this example the suction tube 37 is shown running external to the airway tube and alongside the tube for the inflation means 35 which is used to inflate the cuff assembly 33. this is only one possible arrangement and it will be understood that this suction tube may also run partly within the airway tube if required. It provides a simple means for removing any liquid which collects above the cuff, and is preferably located with an open end at the lowest point above the cuff.

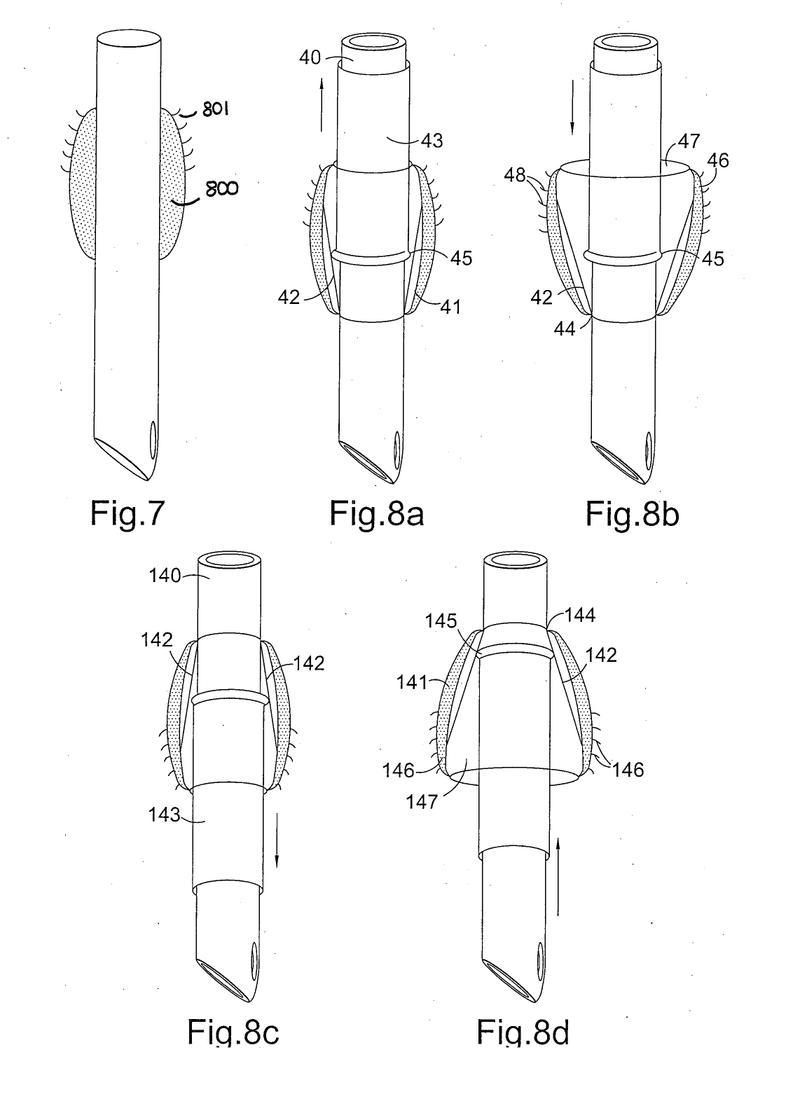

FIG. 7 shows a pediatric airway device incorporating a soft barrier layer 800 over the airway tube but without an inner inflatable region. By choosing the appropriate size of device, an inflatable cuff is no longer required. This applies to adult sizes also. The soft barrier layer 800 is formed from a soft material and incorporates feather-like flanges 801.

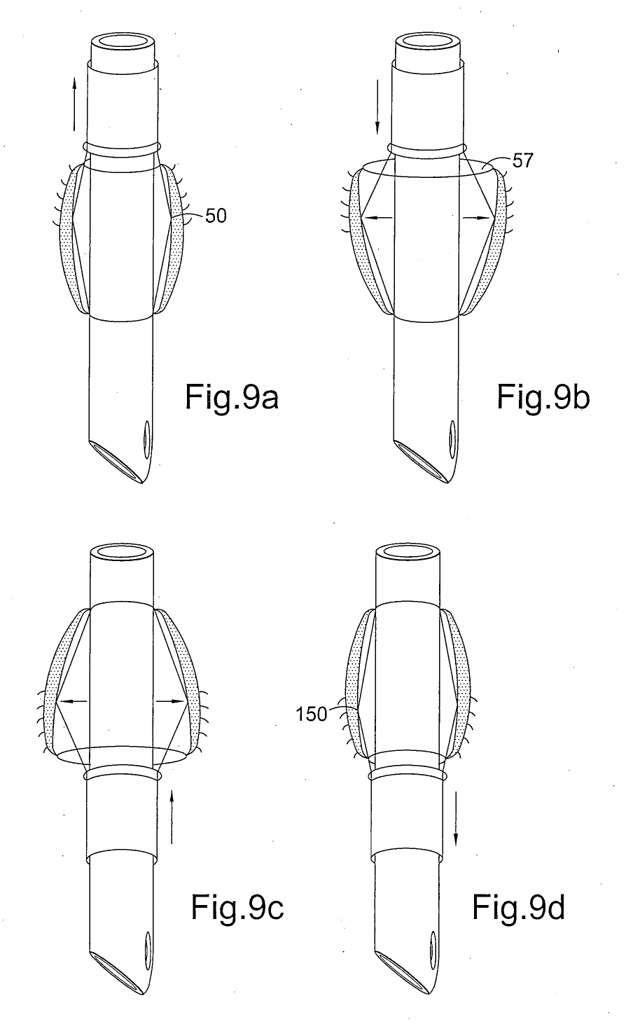

FIGS. 8 and 9 show an alternative embodiment of the present where the cuff assembly is extended using a mechanical extension means. FIG. 8a shows an airway tube 40 ensheathed by a cuff 41. The cuff can be extended (and is shown in an extended position in FIG. 8b) by mechanical extension means comprising an extending portion 42 and an outer sleeve 43. In a non-deployed position as shown in FIG. 8a, the outer sleeve 43, which is a sliding fit with the airway tube, is in a raised position and the extension means does not extend significantly radially out from the tube. The cuff is therefore not extended and the device can be inserted into the airway of a patient. In FIG. 8b, the outer sleeve has been lowered. This has the effect of pushing the extending portion 42, which is pivotally attached to the airway tube at its distal end 44, outwards, extending the cuff, causing the cuff to engage with the walls of the airway (not shown). In this example, the outer sleeve incorporates a ridge 45 at its distal end to facilitate this outwards movement. The outer sleeve thus drives a wedge between the soft material 46 surrounding the tube and the tube 40 itself, forcing the cuff outwards and forming a seal between the cuff and the patient's airway.

The extending portion in this example takes the form of a plurality of fingers arranged circumferentially around the airway tube, much like the spokes of an umbrella. The soft cuff therefore opens and closes much like an umbrella does, in an umbrella-like action.

Because the soft material 46 is not attached to either the tube or the extending portion at its proximal end but is at its distal end sealingly attached to the tube, a cavity 47, see FIG. 8b, is formed between the outer barrier and the tube that is closed at the distal end. In use, aspirate can collect in this region and will not pool at the edges of the cuff. Therefore, less aspirate will be able to pass by the cuff and the aspirate that does pool in the cuff can be easily collected by a suction tube placed at the bottom of the inner cavity (not shown).

Feather flanges 48 are shown on the outside face of the soft material of the cuff. These are an optional feature and help to reduce aspirate passing the cuff and increases the seal that is formed in use between the trachea and the device.

Obviously a moveable outer sleeve is not the only way in which the extending portion can be deployed and any mechanical extension means that forces the cuff away from the tube can be employed. Examples of suitable mechanical extension means include:— an inflatable balloon, inflatable cuff or inflatable annular tube or collar around the lumen; one or more resilient biasing means, biased to hold the cuff away from the airway tube; a moveable device which in a first, closed configuration holds the cuff close to or against the airway tube and in a second, expanded configuration holds the cuff away from the airway tube and against the trachea when in use. Such a moveable device can include some slideable member such as a sleeve, a wire or a string or series of wires/strings, or a spring member or series of spring members. These could be articulated as in the spokes of an umbrella. These examples are not intended to be exhaustive but simple to illustrate the wide variety of extension means that are possible.

The extending portion in this example can be formed from a single component that extends around substantially the entire circumference of the tube or alternatively it can comprise a plurality of fingers that can extend outwards from the tube. The combined radial pressure formed on the outer soft material is sufficient to form a seal with the wall of the airway.

An alternative embodiment is shown in FIGS. 8c and 8d. In this example, which works on similar principles to the version shown in FIGS. 8a and 8b above, the orientation of the cuff is reversed. That is to say, the umbrella-shaped cuff now points downwards, in use, towards the patient's lungs. This simplifies removal of the device at the end of the procedure. Other details remain essentially the same and movement of the ridge 145 causes the cuff to expand away from the airway tube.

FIGS. 9a, 9b, 9c and 9d show an alternative deployment means wherein the extending portion is hinged 50, 150 and is pivotally attached to the tube at its distal end and also pivotally attached to the outer sleeve at its proximal end. It can be seen that the same inner cavity 57 is formed as in the previous example.

The mechanical extension means according to the present invention can be used where the cuff shape is of an umbrella; either a regular, concentric, oval shape or an offset shaped cuff where the conical tip of the umbrella faces distally and the open, circular end faces proximally. A barrier layer with featherlike flanges can be arranged in similar fashion as described in FIG. 1-7. The conical shape of the cuff helps to accumulate any mucous, blood or gastric aspirate. An integral, unitary or a parallel aspiration channel is incorporated to suction out any such accumulated fluid.

The outer sleeve is slideably mounted with respect to the airway tube such that the two concentric components can slide or rotate over each other with a locking and unlocking mechanism towards the proximal end of the tube. The downward and upwards sliding movement of the outer sleeve over the airway tube opens and closes the umbrella shaped cuff and this arrangement avoids the need for a pilot balloon for inflation purposes. This not only reduces the possibility of compression trauma from the balloon but also helps reduce the costs involved in the manufacturing process. This assembly system may include an integral, unitary, parallel or an integrated aspiration/suction channel above the cuff, below the cuff, both above and below the cuff or from the inside of the cone shaped arrangement of the umbrella of the cuff to help suction any material accumulated that collects above and/or below the cuff (not shown).

A monitoring device may be provided at the proximal end of the cuff to give a coloured or other visual monitoring of the cuff expansion while in situ when the tube is slid up and down to open and close the umbrella shaped configuration of the cuff. This monitoring device helps inform the user when a sufficient seal has been formed but any device that serves this purpose is intended to be included within the scope of the present invention.

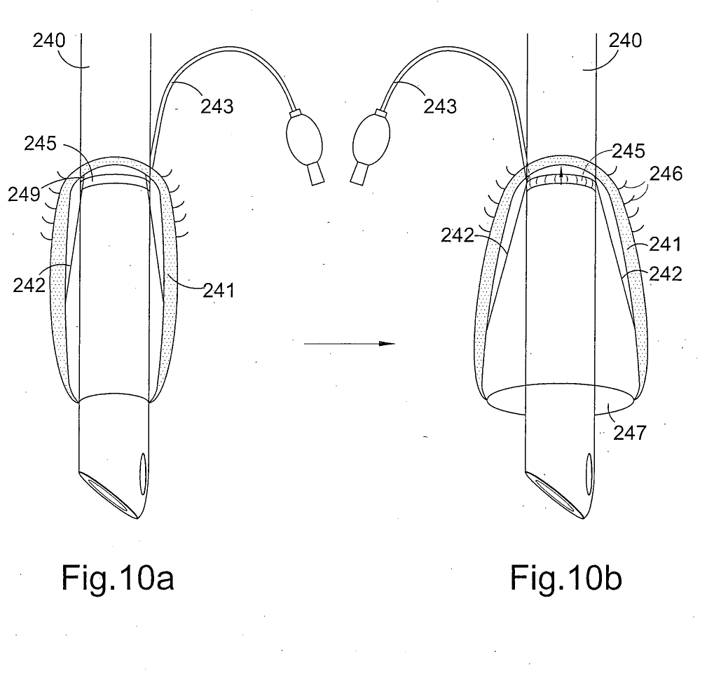

Further embodiments of the present invention are illustrated in FIGS. 10 to 18 inclusive. Once again, they are not intended to be exhaustive examples, but rather to illustrate the wide variety of cuff designs that are possible within the scope of the present invention.

FIGS. 10a and 10b illustrate an alternative embodiment of FIGS. 8 and 9 in which a soft, non-inflatable cuff 241 is activated and expanded by inflation of a small annular collar or balloon 245. The small annular collar or balloon 245 is a further variant of the mechanical extending means to extend the cuff in use into contact with the trachea of the patient. The cuff is formed from a collapsible shroud or substantially frustoconical shaped array of a very soft material. This is supported by a plurality of arms or braces 242 formed of a slightly stiffer or firmer material. These arms are pivotally mounted at one end 249 to the airway tube and are free to move away from or towards the airway tube at the other end. As the collar 245 is inflated the arms 242 are forced away from the airway tube and thus expand the cuff structure, as an umbrella would open. In the context of this disclosure the term “plurality” means one or more.

A key feature of this embodiment is that, whilst it incorporates an inflatable component in the form of a collar, that collar is so sized, shaped and positioned such that it never comes in contact with the patient's trachea during use. Thus, the inflation pressure of the collar, however great that inflation pressure may be, is never transmitted directly to the lining of the trachea.

In other words, the inflatable portion of the cuff assembly of this device is designed not to come into contact with the patient's trachea. Furthermore, there is always an air gap 247, created by free air space, between the airway tube and the non-inflatable cuff of this embodiment in use. This further ensures that minimal damage is caused to the lining of the trachea.

When the inflatable collar 245 is deflated, the arms 242 are biased to collapse the cuff against the sides of the airway tube.

The end of the cuff closest to the inflatable collar is sealed to the airway tube, either directly or indirectly, in a substantially fluid-tight fashion, ensuring that neither air, anaesthetic gas nor liquid can pass by the cuff in that region of the airway tube.

It is also envisaged that the arms or fingers 242 may be attached to the outside of the collar 245, rather than attached directly to the airway tube 240.

It follows therefore that in the example illustrated in FIGS. 10a and 10b, the mechanical extension means comprises an inflatable component. However, this inflatable component is so sized, shaped and configured that it does not come into direct contact with the patient's trachea during normal use. The inflatable component may be an annular collar, extending substantially around the circumference of the airway tube. Alternatively, any other shape of inflatable component which achieves the same end result is intended to fall within the scope of this disclosure and this invention. Such an example might be a series of small, inter-connected balloons, each operating one or more pivotal arms.

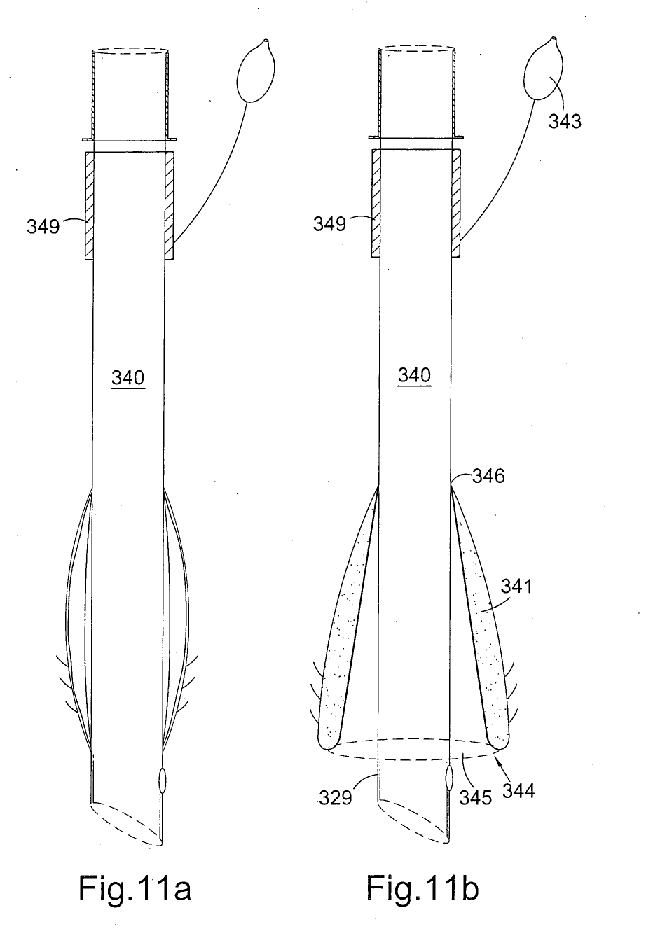

An example of a further embodiment is shown in FIG. 11. Once again, a generally conical shaped cuff is provided which is designed to move between a collapsed configuration, shown in FIG. 11a, and an expanded configuration as shown in FIG. 11b. Here the cuff is inflatable, and the act of inflation causes the cuff 341 to expand, and to be displaced away from the airway tube at one end, 344. The cuff is anchored or attached to the airway tube, either directly or indirectly, at one end 346. The other end of the cuff 344 is free, although constrained in its degree of movement because of the generally frustoconical shape of the cuff itself.

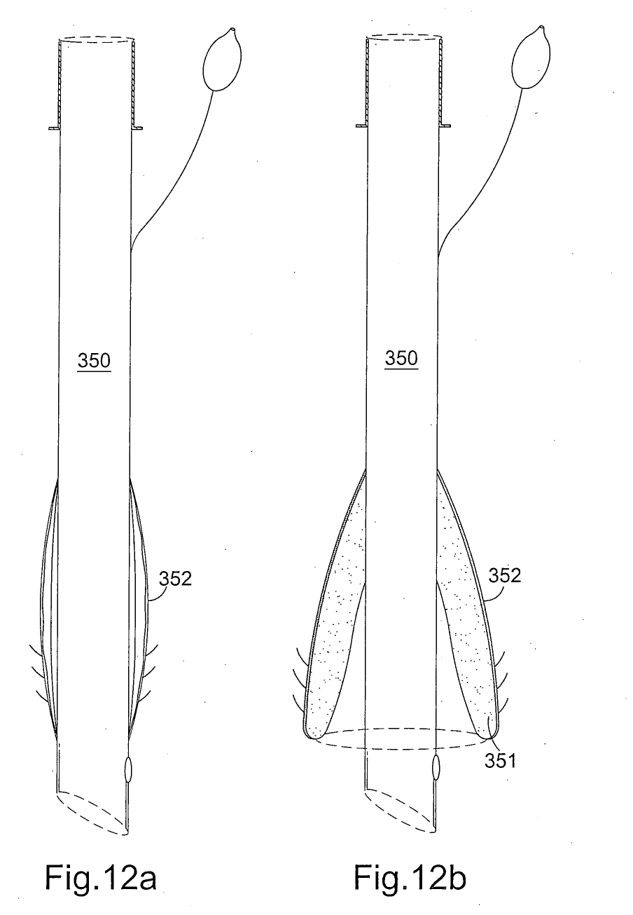

There are a number of specific advantages to this design. Firstly, however hard the cuff is inflated, there is still free air space 345 between the cuff and the airway tube. Thus, the fully inflated cuff cannot cause damage to the patient's trachea in the way that a standard inflatable endotracheal cuff does. Furthermore, the inflatable portion of the cuff can have an outer coating of very soft material, such as a material having a Shore hardness on a scale of between 000 to 40. Such a soft, barrier layer is shown in FIG. 12 as 352.

A further important but not essential feature is shown in FIG. 11. This is a bite block 349, designed to prevent the patient biting onto the airway tube and compressing it, thus cutting off his/her oxygen supply. The bite block is formed by a region of rigid plastic or other material that is harder to compress than the generally soft airway tube itself. In this example the bite block is formed from a slideable sleeve which is a tight sliding fit over the outside of the airway tube, and which can be positioned at the appropriate point or region by the operator. A bite block of this type is applicable to any of the embodiments described herein.

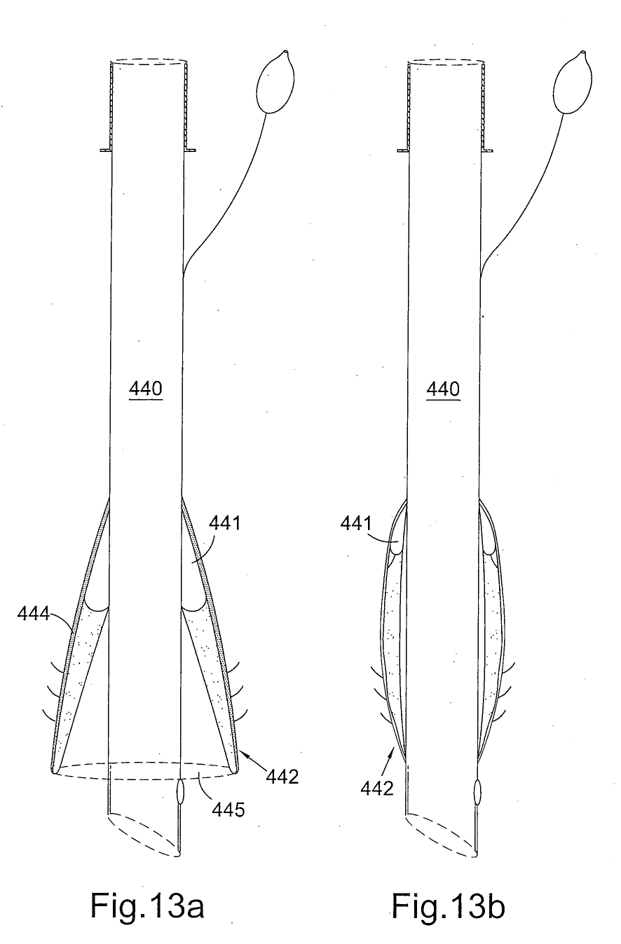

A further variation is shown in FIG. 13 where a small inflatable balloon 441 is used to expand a non-inflatable but expandable cuff 442 against the tracheal wall of the patient, in use. This principle, where an inflatable balloon is used as a mechanical extension means to expand an otherwise non-inflatable cuff to form a seal with the tracheal wall, is an important advance. It offers several advantages over the prior art arrangements. In particular, the maximum inflation pressure of the balloon 441 is never transmitted directly to the tracheal wall, simply because the inflated balloon never comes in contact with the tracheal wall itself. Additionally, the surface of the cuff 444 where it contacts the trachea is coated with a very soft material, to minimise the possibility of damage to the tracheal wall.

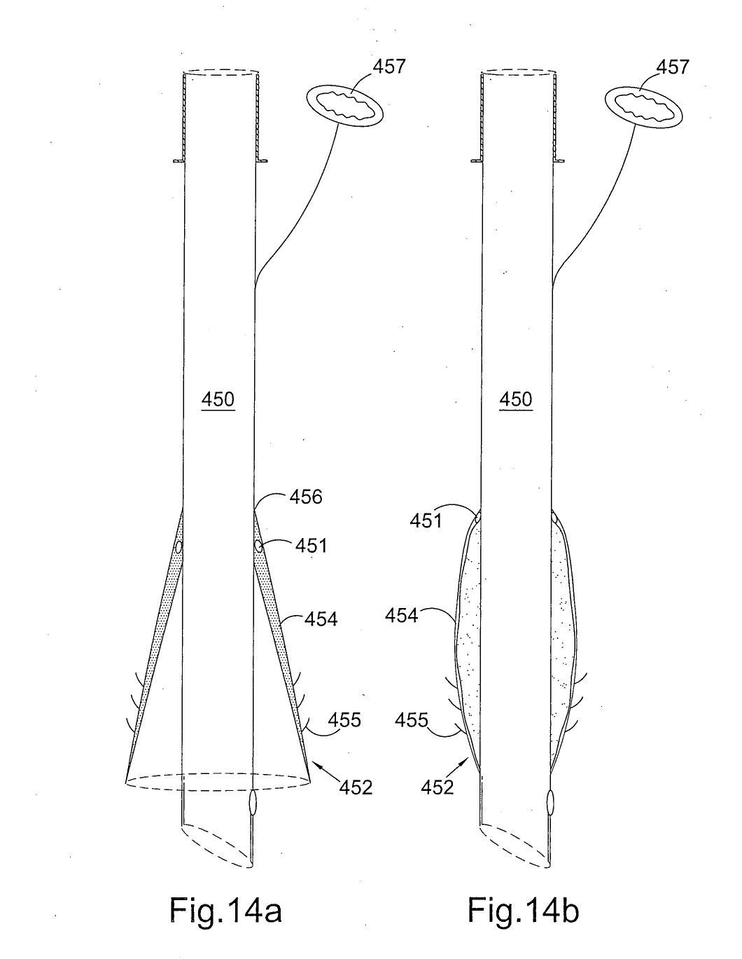

A further variation is shown in FIGS. 14a and 14b where a hook and cord 457 is used to deploy the cuff assembly 452 from an undeployed position as shown in FIG. 14b to a deployed position as shown in FIG. 14a. The hook 457 is connected to the cuff 451 such that as the hook 451 is pulled upwards the cuff 452 becomes deployed.

In this embodiment, levers 451 in the form of arms are deployed as part of the cuff assembly. Putting the hook and cord under tension causes these levers to pivot outwards, about points 456 at one end of each lever. This pivotal movement causes the cuff assembly to open in an umbrella-like manner. A soft barrier layer 454 both improves the seal with the trachea and protects the trachea from damage.

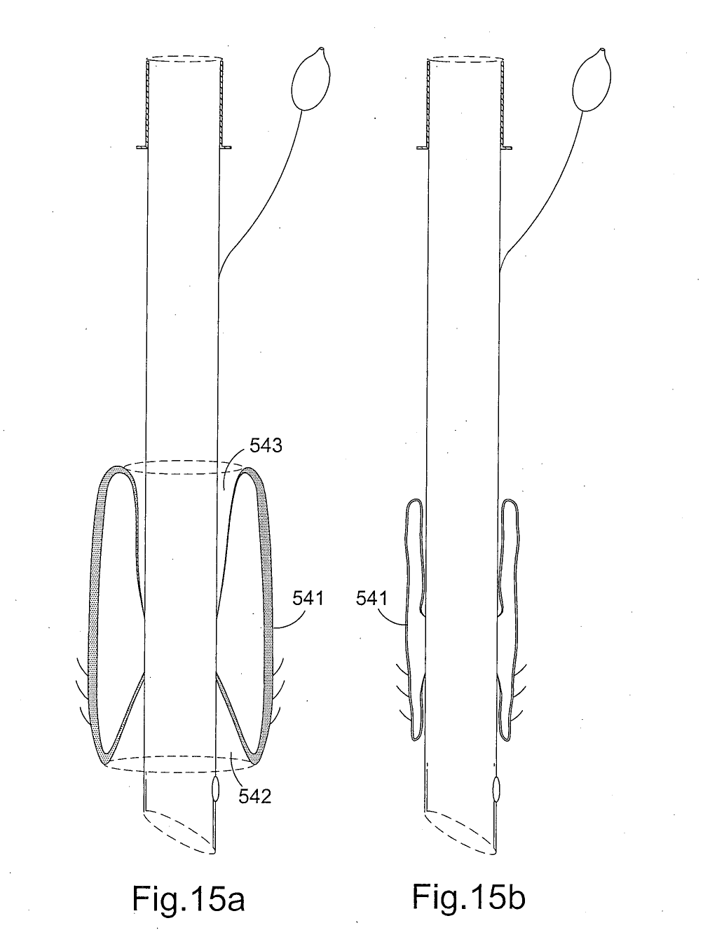

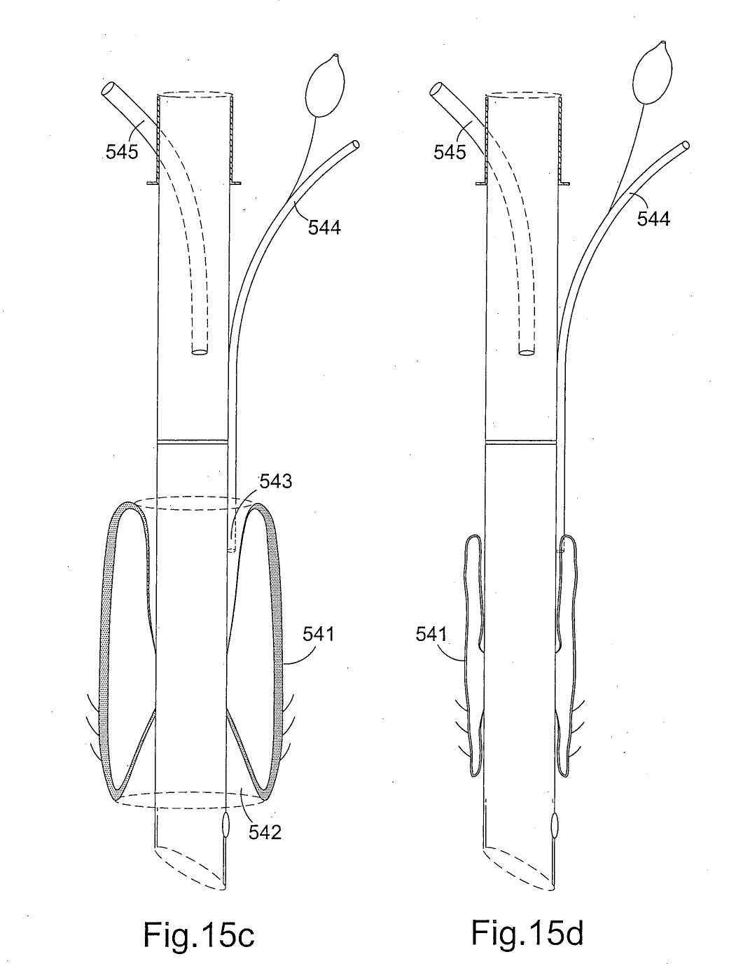

Further aspects of the invention are shown in FIG. 15. In FIG. 15, a butterfly-shaped inflatable cuff 541 is used. This allows for a free air gap 542, 543 between the inner face of the cuff and the airway tube. This minimises the possible trauma caused by the cuff, even if it is inflated to a high operating pressure.

FIGS. 15c and 15d show a further alternative of FIGS. 15a and 15b which can be further incorporated into any of the other devices disclosed herein. The device is provided with a gastric channel 545 through which a gastric tube may be passed which will continue down the endotracheal airway tube and out of the distal tip to remove gastric fluids either directly from the stomach or any regurgitated and pooling around the lower cuff seal. The device may further incorporate a suction channel 544 for removal of secretions from the airway device pooling around the upper cuff seal.

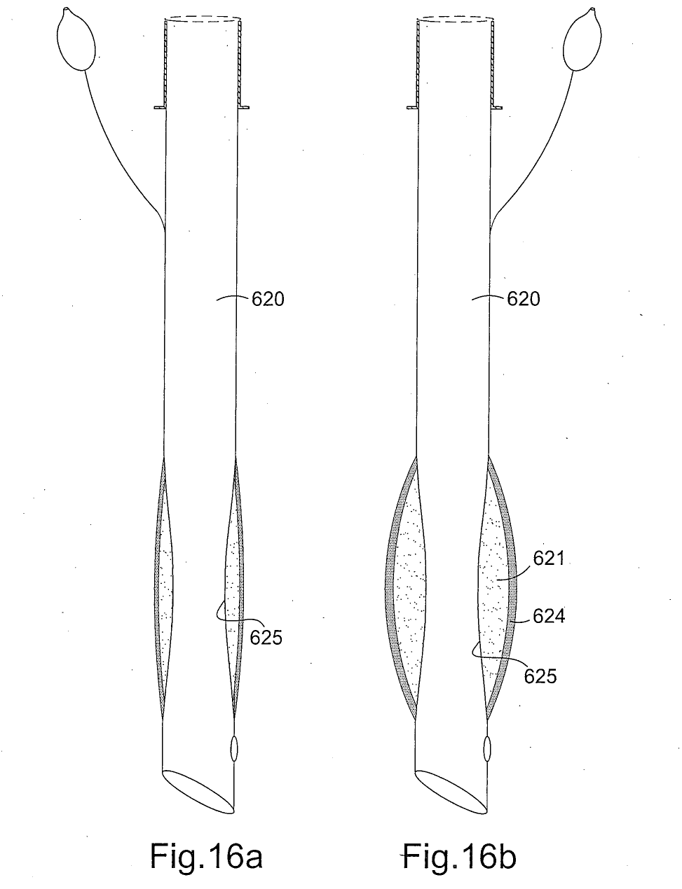

A further important feature of this invention, applicable to all aspects and embodiments is shown in FIG. 16. This shows an airway tube 620 which is a substantially constant internal diameter over the majority of its length. However, in the region of the cuff, this diameter is reduced, and the external diameter of the tube is also reduced. In this way part or all of the body of the cuff assembly may be accommodated within the overall external diameter of the endotracheal tube. The result is that the tube, with the cuff in its non-expanded configuration, is generally more streamlined and is easier to insert into the trachea. The external face of the airway tube thus incorporates a depression, manifested as a region of reduced external diameter, to accommodate some or all of the cuff structure.

The principle of reducing the outside diameter of the airway tube in the region of the cuff is applicable to all of the embodiments described herein. This provides the significant advantage that the volume of the cuff in its collapsed state can be accommodated substantially within a profile that is no larger than the diameter of the airway tube along most of its length.

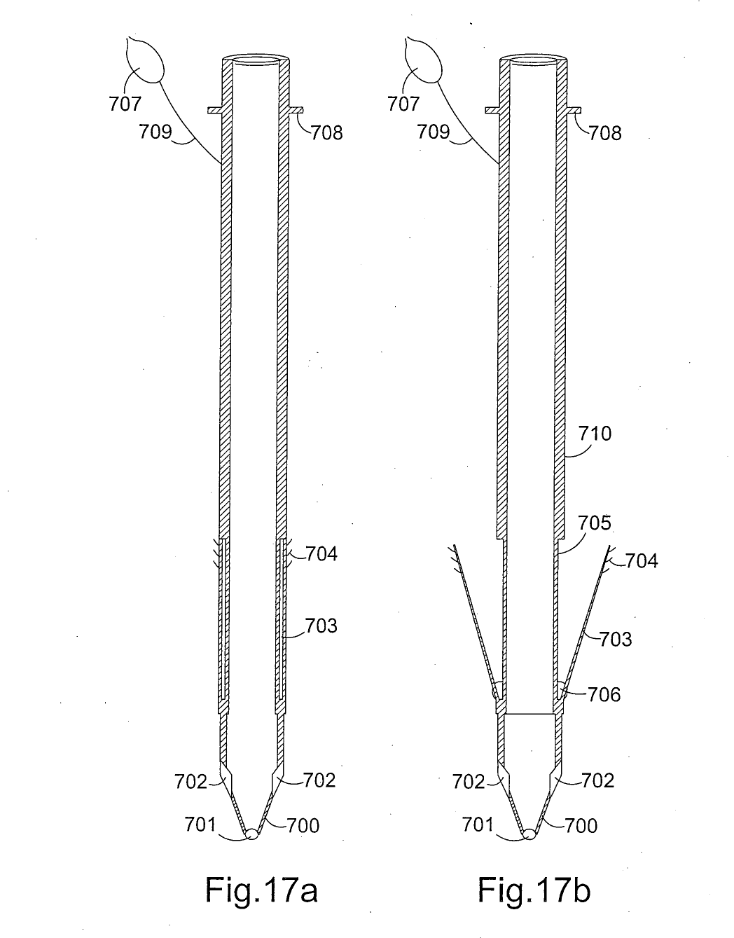

FIGS. 17a and 17b show an alternative distal tip arrangement which is a different configuration to the standard distal tip of endotracheal tubes as known in the art and as shown in the previous figures. In this embodiment, the distal tip 700 is tapered symmetrically on all sides to a hollow point 701 substantially in the centre of the distal tip 700. The tip of the airway tube is further provided with apertures 702 located around the endotracheal tube disposed near the distal tip 700 around the point where the distal tip 700 begins to taper to its hollow point 701. This alternative distal tip arrangement is beneficial as the hollow point 707 is in the centre of the endotracheal airway tube 710 and thus less likely to catch on the architecture of the human or animal body during insertion. This alleviates or eliminates a problem found with conventional endotracheal tubes. Once again, this modification is applicable to all the embodiments described herein.

The mechanical extension means of the cuff 703 is an alternative embodiment to that shown in FIGS. 10 and 13. The cuff 703 in a non-deployed position is shown in FIG. 17a and the cuff 703 in a deployed position is shown in FIG. 17b. Air is injected into an injection port 707 and travels down an inflation line 709 to a deployment means 706 which inflates causing the cuff 703, which is pivotally connected to the endotracheal airway tube 710, to be deployed and to open up like an umbrella. The endotracheal airway tube is further provided in one alternative with a recess 705 which has the same dimensions as the cuff 703 in an undeployed state such that when the cuff 703 is not deployed, the cuff 703 fits into the recess 705 for ease of insertion of the device. The cuff, in one alternative, may be provided with feather flanges 704 to assist in sealing the endotracheal airway tube 710 with the human or animal anatomy.

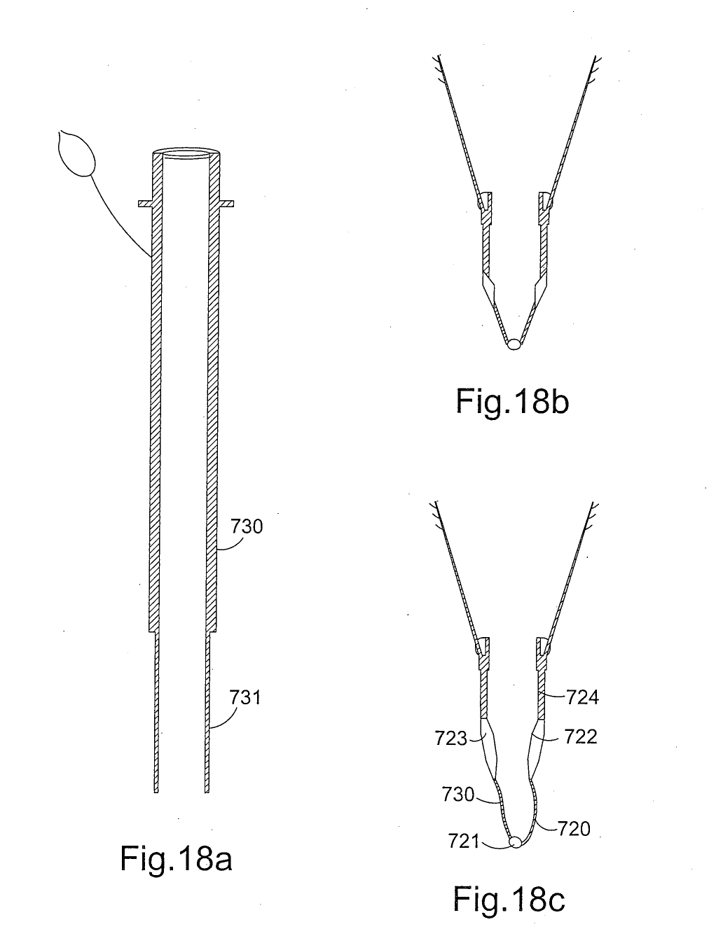

In an alternative shown in FIGS. 18a, 18b and 18c, this illustrates how a different distal tip arrangement can be employed. In FIG. 18b, the same distal tip 700 is employed as in FIGS. 17c and 17b. FIG. 18c, however, shows an alternate distal tip assembly 720 which is further shaped to avoid the architecture of the human or animal during insertion. Advantageously, the opening in the hollow tip 721 is still formed substantially central to the endotracheal airway tube portion 724. On one side of the distal tip 720, the airway tube is generally straight but inset towards the centre of the airway tube and on the opposing side the distal tip 720 is significantly curved.

In this arrangement, the top of the device, which is narrower than the general external diameter of the airway tube, is bent around, much as the end of a finger may be bent. In effect, the longitudinal axis of the distal tip of the device is offset from the longitudinal axis of the airway tube. One side of the distal tip 730 is substantially planar whilst the opposing side 720 is substantially curved such that it is slightly convex when viewed from that side. The result of this is that on the curved side part of the outside of the distal tip is substantially aligned with the body of the airway tube, before curving inwards towards the longitudinal axis of the device. This arrangement further prevents the open tip of the device 721 coming into contact with the inside of the patient's airway.

FIG. 18a, in conjunction with FIGS. 18b and 18c, shows a novel form of construction of an airway device. In this embodiment, an airway tube 730 is formed of plastics material having a conventional thickness. The distal end of this tube 731 is formed from a region of a small outside diameter. This smaller outside diameter 731 fits inside the tip region 724 such that the cuff assembly is also accommodated within the recess shown as 705 in FIG. 17b. The tip assembly and the airway tube portion can be joined together by known procedures such as ultrasonic welding or adhesive or solvent bonding to give a unitary construction. The outside of the airway tube, and/or the outside of the distal tip assembly, can be coated with a very soft material, having a Shore hardness on the A scale of 000 to 40, more preferably 000 to 20. Methods of co-extruding or over-moulding plastics of different Shore hardness are known.

All of the airway devices described above can incorporate a suction channel, a suction device or a Murphy eye in the case of an endotracheal tube.

The presence of a Murphey's eye is desirable but not an absolute essential. The distal end of the airway tube can be bevelled or circular. If it is bevelled, then the face of the blunted tip of the bevel is preferably angled by 5 to 10 degrees towards the lumen of the tube, which helps ease of insertion of the tube during intubation.

A suction channel can be provided above the cuff, below the cuff or alternatively, suction channels can be provided both above and below the cuff. In the case of the offset cuff the suction channel is preferably located towards the region 28 as can be seen in FIG. 4a where any aspirate collects. In the case of the mechanically expanded cuff, the channel can remove aspirate pooling inside the outer barrier region of the cuff.

Furthermore, the invention is applicable to airway devices that have a single cuff or a multiple cuff. The barrier layer, offset cuff and offset tube have wide application and are not intended to be limited to the specific examples described above.

The cuff assemblies described herein can also be advantageously used in devices that need to travel through, block or seal any lumen in a patient. For example, the device is applicable to any catheter such as an angioplasty catheter, catheters used for neurosurgery, cardiothoracic, vascular, genitourinary, gynaecological procedures. This list is not intended to be exhaustive but merely shows the wide application of this invention to any catheter or other medical device.

If desired there may be provided reinforcement of the tube with a spring mechanism for the ease of mobilisation of the tube in certain operative procedures of the head and neck. Preformed shapes of the tube could also be desirable for certain operative procedures of the head and neck.

1-54. (canceled)

55. An airway device for insertion into the trachea or bronchi of a human or animal, comprising an airway tube having a distal end, a proximal end, the device further comprising a cuff located at or near the distal end of the flexible tube, wherein the cuff comprises an inflatable collar or balloon operable to move the cuff from a non-extended position wherein the cuff does not engage the walls of the trachea or bronchi and an extended position wherein the cuff is generally conical in shape and engages the walls of the trachea or bronchi.

56. An airway device as claimed in claim 55 wherein when the inflatable collar or balloon is in the deflated rest position the generally conical shaped cuff is in the non-extended position or closed position and when the inflatable collar or balloon is in to the inflated position the cuff moves to the extended position or open position such that the cuff engages the walls of the trachea or bronchi.

57. An airway device as claimed in claim 55 wherein the cuff is anchored or attached to the airway tube either directly or indirectly at a first end of the cuff, a second end of the cuff being free such that there is a space between the second end of the cuff and the airway tube.

58. An airway device as claimed in claim 55 wherein the non-opening end of the generally conical cuff is mounted around the circumference of the airway tube.

59. An airway device as claimed in claim 55 wherein cuff has a proximal end and a distal end and wherein the proximal end of the cuff is open in use to allow aspirate to collect within the cuff.

60. An airway device as claimed in claim 55 wherein the cuff further comprises an outer barrier region and wherein the outer barrier region is formed from a material with a Shore A hardness of 40 or less.

61. An airway device as claimed in claim 55 wherein the distal end of the airway tube further comprises a distal tip wherein the distal tip is formed from or coated with a material with a Shore A hardness of less than 20.

62. An airway device as claimed in claim 55 wherein the distal end of the airway tube further comprises a distal tip wherein the distal tip is tapered symmetrically on all sides to a hollow point.

63. An airway device as claimed in claim 55 wherein the distal end of the airway tube further comprises a distal tip wherein the distal tip comprises a Murphey's eye.

64. An airway device as claimed in claim 55 wherein the cuff incorporates one or more feather like flange(s) having a base attached to the cuff and a tip.

65. An airway device as claimed in claim 55 wherein the cuff is umbrella shaped.

66. An airway device as claimed in claim 55 further comprising a gastric tube passageway or suction channel.

67. An airway device as claimed in claim 55 further comprising a bite block.

68. An airway device as claimed in claim 55 wherein the airway device is an endotracheal tube and/or an endobronchial tube.
